(19) United States
(12) Patent Application Publication
(10) Pub. No.: US 2023/0264986 A1
(43) Pub. Date: Aug. 24, 2023

(54) NANOCOMPOSITE SEPARATION MEDIA AND METHODS OF MAKING THE SAME

(71) Applicant: THE UNIVERSITY OF NORTH CAROLINA AT CHARLOTTE, Charlotte, NC (US)

(72) Inventors: Abhispa Sahu, Charlotte, NC (US); Jordan C. Poler, Charlotte, NC (US)

(21) Appl. No.: 18/020,694

(22) PCT Filed: Aug. 13, 2021
(86) PCT No.: PCT/US2021/045915

§ 371 Date: Feb. 10, 2023

Related U.S. Application Data
(60) Provisional application No. 63/065,669, filed on Aug. 14, 2020.
(60) Provisional application No. 63/190,994, filed on May 20, 2021.

(51) Int. Cl. C02F 1/42; B01J 20/20; B01J 20/26; B01J 20/28; B01J 20/24; B01J 20/32

(52) U.S. Cl. CPC C02F 1/42; B01J 20/205; B01J 20/265; B01J 20/28016; B01J 20/24; B01J 20/3255; B01J 20/3293; C02F 2001/422; C02F 2001/425; B01J 2220/46; B01J 2220/4806; C02F 2101/20

(57) ABSTRACT

Nanocomposite materials are described herein which, in some embodiments, are employed as separation media for removal of various contaminants from water sources, including heavy metals, PFAS and/or NOM. In some embodiments, a nanocomposite material comprises oligomeric chains or polymeric chains covalently attached to surfaces of fluorographite at sites of defluorination. In another aspect, nanocomposite materials based on cellulose nanofibers are described herein. In some embodiments, a nanocomposite material comprises oligomeric chains or polymeric chains covalently attached to surfaces of cellulose nanofibers.

### RELATED APPLICATION DATA

The present application claims priority pursuant to 35 U.S.C. § 119(e) to U.S. Provisional Patent Application Ser. No. 63/065,669 filed Aug. 14, 2020 and to U.S. Provisional Patent Application Ser. No. 63/190,994 filed May 20, 2021, each of which is incorporated herein by reference in its entirety.

### STATEMENT OF GOVERNMENT RIGHTS

This invention was made with government support under grant number 83996101 awarded by the United State Environmental Protection Agency (USEPA). The government has certain rights in the invention.

### BACKGROUND

Natural organic matter (NOM) is comprised of decomposed plant and animal residues, and exists in all active water resources. When NOM levels are too high (e.g., due to the natural or the continued rise of anthropogenic causes), they must be reduced via water treatment methods. NOM compounds, namely humic and fulvic acids, are also sources of potential health hazards to humans and animals that will ingest treated water.

Modern water treatment methods utilize chlorine to destroy microbial pathogens. However, chlorine reacts with humic substances in NOM and forms disinfection byproducts (DBPs) that have deleterious human health risks. Moreover, the two most common regulatory problems stem from trihalomethanes (THMs) and haloacetic acids (HAAs), and subsequent interest has been placed on efficient removal of their precursors.

It is becoming increasingly difficult for water treatment facilities to mitigate the formation of DBPs at the limits set by the USEPA (i.e., 80 ppb for THM and 60 ppb for HAA). Prolonged exposure to DBPs can lead to kidney, liver, and central nervous system damage, as well as cancer. Better treatment technologies are needed to improve water quality and reduce the precursors which lead to the formation of carcinogenic DBPs in drinking water.

Removal of NOM is a primary concern for providing safer, cleaner water, and the extent of its removal depends on the efficiency of the treatment methods employed. Mismanagement of water treatment facilities and poor public policy, acutely punctuated by the events in Flint Mich., exemplify a potential systemic risk to our society. Even with the practice of “enhanced coagulation” as prescribed by the USEPA, a source water with 2 mg/L of dissolved organic carbon (DOC) and a moderate alkalinity of 60-120 mg/L would only be required to remove 25% of the total organic carbon present in the water.

Many water treatment facilities rely solely on coagulation as the means of lowering levels of DOC; however, this is not an effective method of removing low molecular weight and hydrophilic varieties of NOM, as such smaller molecules are more readily removed via adsorption.

Some treatment facilities are looking into utilizing activated carbon to adsorb NOM, however; this method comes with a high operating cost. With increasing concentrations of NOM being observed in drinking water sources worldwide, there has been a significant increase in demand for more efficient removal.

Additionally, per- and polyfluoroalkyl substances (PFAS) are emerging contaminants present in many consumer goods. These fluorochemicals are of significant concern due to their potential health effects. Because of their high water solubility, they are ubiquitous in drinking water sources, including groundwater, which becomes the main source of exposure to humans. Efforts in sustainable manufacturing of chemical compounds require that compounds for release into the environment are degradable. PFAS are very stable and little is known about their biodegradability. Even less is known about their mineralization (complete biodegradation to CO₂, IF and water, etc).

Release of polyfluoroalkyl chemicals into the environment can result in the formation of perfluoroalkyl carboxylic (PFCAs) and sulfonic acids (PFSAs), such as perfluorooctanoic acid (PFOA) and perfluorooctane sulfonic acid (PFOS). These compounds are highly persistent and detected widely in the environment. It is unclear if these smaller moieties can be mineralized and, so far, a lack of mineralization data has been reported. Moreover, multiple studies on the degradation of various PFAS concluded that these compounds are stable in the environment.

Accordingly, a need exists for improved materials, devices, and methods of removing NOM, PFAS and other contaminants from liquids, such as water, and for providing safer drinking water having reduced levels of DBPs and PFAS.

### SUMMARY

In view of the foregoing, nanocomposite materials are described herein which, in some embodiments, are employed as separation media for removal of various contaminants from water sources, including heavy metals, PFAS and/or NOM. In some embodiments, a nanocomposite material comprises oligomeric chains or polymeric chains covalently attached to surfaces of fluorographite at sites of defluorination. The oligomeric chains or polymeric chains, for example, can conformally coat individual platelets of the fluorographite. In doing so, the oligomeric chains or polymeric chains are present in spacings or locations between individual platelets of the fluorographite. Moreover, in some embodiments, the fluorographite functionalized with the oligomeric or polymeric chains exhibits a fluorine content of less than 40 at. %, such as 5-20 at. % fluorine.

As described further herein, the functionalized fluorographite can be employed in separation media or filtration media. In some embodiments, a separation medium comprises a stationary phase including oligomeric chains or polymeric chains covalently attached to surfaces of fluorographite at sites of defluorination. The oligomeric chains or polymeric chains, in some embodiments, comprise one or more cationic moieties for anion exchange. Alternatively, the oligomeric chains or polymeric chains can comprise anionic moieties for cation exchange. In further embodiments, the oligomeric chains or polymeric chains comprise fluorinated moieties, such as fluorinated aryl and/or fluorinate alkyl moieties for PFAS removal from water sources.

In another aspect, methods of functionalizing fluorographite are provided. A method, in some embodiments, comprises providing a reaction mixture including fluorographite and an oligomeric species or polymeric species. Surfaces of the fluorographite are covalently functionalized with the oligomeric species or polymeric species via a radical reaction mechanism. The oligomeric species or polymeric species, for example, can comprise a radical end prior to covalently binding to sites of defluorination. Moreover, the reaction mixture can comprise an aqueous continuous phase. In some embodiments, the fluorographite is exfoliated via sonication or other means prior to covalent functionalization with the oligomeric or polymeric chains. Additionally, in some embodiments, the reaction mixture does not comprise one or more external agents for defluorination of the fluorographite.

In another aspect, methods of treating water are described herein. In some embodiments, a method comprises providing a separation medium including a stationary phase comprising oligomeric chains or polymeric chains covalently attached to surfaces of fluorographite at sites of defluorination. The separation medium is contacted with a water source, and one or more contaminants are removed from the water source by the stationary phase of the separation medium. Separation media described herein can be regenerated subsequent to contaminant removal from the water source. In some embodiments, the separation medium does not exhibit performance losses for contaminant removal after 20 or 30 regeneration cycles. In some embodiments, for example, the separation medium loses less than 1%, less than 0.5% or less than 0.1% contaminant adsorption performance over at least 40 cycles of regeneration.

In another aspect, nanocomposite materials based on cellulose nanofibers are described herein. In some embodiments, a nanocomposite material comprises oligomeric chains or polymeric chains covalently attached to surfaces of cellulose nanofibers. The oligomeric chains or polymeric chains, for example, can be in spacings or locations in between individual cellulose nanofibers. As described above, the oligomeric chains or polymeric chains can comprise cationic, anionic, or fluorinated moieties. Chemical identity of the oligomeric chains or polymeric chains can be selected on the type of contaminant to be removed from a water source. Accordingly, the covalently functionalized cellulose nanofibers can be employed in water filtration or purification applications. The cellulose nanofibers can serve as a stationary phase of the separation medium.

In another aspect, methods of making cellulose-based nanocomposite materials are described herein. A method, in some embodiments, comprises providing cellulose nanofibers, and oxidizing the nanofibers to provide reactive surface sites. Oligomeric species or polymeric species are provided and covalently attached to the reactive surfaces sites via esterification or a radical reaction mechanism. In some embodiments, the esterification or radical reaction mechanism occurs in an aqueous reaction medium.

These and other embodiments are further described in the detailed description which follows.

### BRIEF DESCRIPTION OF THE DRAWINGS

FIG. 1 illustrates quaternary ammonium cationic moieties of oligomeric or polymeric chains according to some embodiments.

FIG. 2 and FIG. 3 illustrate methods of producing cellulose nanofibers covalently functionalized with oligomeric chains or polymeric chains via esterification, according to some embodiments.

FIG. 4A provided ATR FTIR spectra of fluorographite and PFG-V demonstrating defluorination and functionalization of poly(vbTMAC) on fluorographite according to some embodiments.

FIG. 4B illustrates XPS survey scan of fluorographite, PFG-I and PFG-V, according to some embodiments.

FIG. 4C provides high resolution C is spectra fluorographite and deconvolution results.

FIG. 4D provides high resolution C is spectra PFG-V and deconvolution results.

FIG. 5 provides measured EELS spectra showing F—K edge of fluorographite and PFG-V exhibiting characteristic post peak at 700 eV supporting defluorination processes.

FIG. 6A is a transmission electron micrograph of fluorographite sheets.

FIG. 6B is a transmission electron micrograph of PFG-V according to some embodiments.

FIG. 6C is a SAED pattern of PFG-I according to some embodiments.

FIG. 6D is a SAED pattern of PFG-V according to some embodiments.

FIG. 7 illustrates XRD patterns of pristine fluorographite and PFG-I, according to some embodiments.

FIGS. 8A and 8B illustrate Raman spectra of fluorographite and PFG-V, respectively.

FIGS. 9A and 9B are scanning electron micrographs of a PFG-V thin film revealing nanoplate-like morphology according to some embodiments.

FIG. 10 illustrates kinetic studies of PFG-V using NaFL as analyte according to some embodiments.

FIG. 11 illustrates adsorption isotherms of PFG-V according to some embodiments.

FIG. 12 illustrates percent removal of emerging contaminants by PEG-I as a function of mass density (mg cm⁻²) of PFG thin film deposited on MCE membrane according to some embodiments.

FIG. 13 quantifies regeneration and reusability studies of PFG-V thin films using NaFL as surrogate, according to some embodiments.

FIG. 14 illustrates adsorption isotherm analysis for covalently functionalized cellulose nanofibers according to some embodiments.

FIG. 15 illustrates a synthetic scheme for producing cellulose nanofibers covalently functionalized with oligomeric chains or polymeric chains.

### DETAILED DESCRIPTION

Embodiments described herein can be understood more readily by reference to the following detailed description, examples, and figures. Nanomaterials, supports, devices, and methods described herein, however, are not limited to the specific embodiments presented in the detailed description, examples, and figures. It should be recognized that these embodiments are merely illustrative of the principles of this disclosure. Numerous modifications and adaptations will be readily apparent to those of skill in the art without departing from the disclosed subject matter.

All ranges disclosed herein are to be understood to encompass any and all subranges subsumed therein. For example, a stated range of “1.0 to 10.0” should be considered to include any and all subranges beginning with a minimum value of 1.0 or more and ending with a maximum value of 10.0 or less, e.g., 1.0 to 5.3, or 4.7 to 10.0, or 3.6 to 7.9.

Further, all ranges disclosed herein are also to be considered to include the end points of the range, unless expressly stated otherwise. For example, a range of “between 5 and 10” or “5 to 10” or “5-10” should generally be considered to include the end points 5 and 10.

Additionally, in any disclosed embodiment, the terms “substantially,” “approximately,” and “about” may be substituted with “within [a percentage] of” what is specified, where the percentage includes 0.1, 1, 5, and 10 percent.

The terms “a” and “an” are defined as “one or more” unless this disclosure explicitly requires otherwise. The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a result, a composition or other object that “comprises,” “has,” “includes” or “contains” one or more elements possesses those one or more elements, but is not limited to possessing only those elements. Likewise, a method that “comprises,” “has,” “includes” or “contains” one or more steps possesses those one or more steps, but is not limited to possessing only those one or more steps.

It is further understood that the feature or features of one embodiment may generally be applied to other embodiments, even though not specifically described or illustrated in such other embodiments, unless expressly prohibited by this disclosure or the nature of the relevant embodiments. Likewise, materials, devices, and methods described herein can include any combination of features and/or steps described herein not inconsistent with the objectives of this disclosure. Numerous modifications and/or adaptations of the materials, devices, and methods described herein will be readily apparent to those skilled in the art without departing from the subject matter described herein.

### I. Covalently Functionalized Fluorographite

In one aspect, nanocomposite materials based on fluorographite are described herein. In some embodiments, a nanocomposite material comprises oligomeric chains or polymeric chains covalently attached to surfaces of fluorographite at sites of defluorination. The oligomeric chains or polymeric chains, for example, can conformally coat individual platelets or layers of the fluorographite. In doing so, the oligomeric chains or polymeric chains are present in spacings or locations between individual platelets or layers of the fluorographite.

The covalently functionalized fluorographite can exhibit nanoplatelet morphology comprising 10 to 20 layers. The layered nanoplatelets can have thickness or diameter of 50 nm to 100 nm, in some embodiments. Particles or nanoplatelets of covalently functionalized fluorographite can adopt stacking arrangement in the formation of thin films. In some embodiments, stacking of the oligomeric or polymeric functionalized fluorographite can provide thin films of 500 nm to 1 μm in thickness. These films can be applied to polymeric, ceramic and/or metal supports as separation media in the fabrication of water filtration/purification apparatus.

As described further herein and illustrated in the following examples, covalent functionalization of the fluorographite occurs at sites of defluorination. Accordingly, fluorographite covalently functionalized with oligomeric or polymeric chains contains less fluorine than native or pristine fluorographite. Fluorine content, for example, can generally be directly proportional to degree of surface functionalization by the oligomeric chains or polymeric chains. In some embodiments, covalently functionalized fluorographite described herein has a fluorine content less than 40 at. %. Fluorine content of the covalently functionalized fluorographite can also have a value selected from Table I, in some embodiments.

TABLE I
Fluorine Content of Covalently Functionalized
Fluorographite (at. %)
5-38
5-30
10-20
10-15
5-15
5-10

Fluorine content of the covalently functionalized fluorographite can be determined according to X-ray photoelectron spectroscopy (XPS).

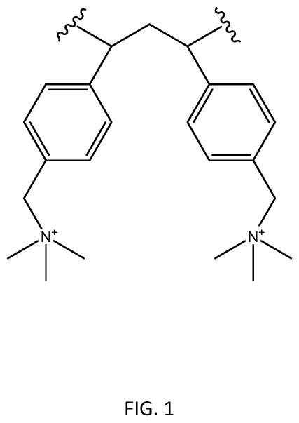

The oligomeric chains or polymeric chains covalently attached to the fluorographite at site of defluorination can include one or more cationic moieties for anion exchange. The cationic moieties can have compositional identity to provide a strong anion exchange medium or weak anion exchange medium. Cationic moieties, for example, can comprise quaternary ammonium groups or imidazolium groups. FIG. 1 illustrates quaternary ammonium cationic moieties 106 of oligomeric or polymeric chains according to some embodiments. Alternatively, the oligomeric chains or polymeric chains can comprise one or more anionic moieties for cation exchange. Anionic moieties can have compositional identity to provide strong cation exchange medium or weak cation exchange medium. In some embodiments, for example, anionic moieties include sulfonic acid groups, carboxylic acid groups and corresponding salts and/or derivatives thereof. In further embodiments, oligomeric chains or polymeric chains can be neutral, wherein charged moieties are absent on the oligomeric chains. In some embodiments, the oligomeric chains or polymeric chains comprise fluorinated moieties. Fluorinated moieties can include fluoroalkyl and/or fluoroaryl moieties, such as perfluorinated and polyfluorinated alkyl and aryl moieties. The oligomeric chains or polymeric chains covalently attached to surfaces of the fluorographite are not crosslinked, in some embodiments. Moreover, the oligomeric chains can comprise 3-100 monomeric units, in some embodiments. In some embodiments, the oligomeric chains or polymeric chains comprise a mixture of cationic moieties and fluorinated moieties. The ratio of cationic moieties to fluorinated moieties can vary from 100:1 to 1:100. Additionally, in some embodiments, cationic, anionic and/or fluorinated moieties are located on groups pendant to the oligomeric chain or polymeric chain backbone.

As described herein, the fluorographite covalently functionalized with oligomeric chains or polymeric chains can employed as the stationary phase in water filtration/purification apparatus and applications. In some embodiments, the functionalized nanoplatelets of fluorographite can be assembled into thin films for removing contaminants from a water source. The functionalized fluorographite films can have any desired thickness. In some embodiments, the fluorographite thin films have thickness of 500 nm to 1 μm. In other embodiments, thickness of the functionalized fluorographite layers can be greater than 1 μm. The films can be placed on any desired support, including polymeric supports, ceramic supports, and metallic supports. In some embodiments, a support for the functionalized fluorographite layers is mixed ester cellulose.

Depending on specific chemical identity, the oligomeric chains or polymeric chains can remove heavy metals, NOM, PFAS substances, oxyanions, and/or contaminants containing humic acid, fulvic acid, trihalomethane, haloacetic acid, a carboxylate group, or a phenolate group. In some embodiments, PFAS compounds include those listed in the following table.

Heptafluorobutyric acid (HFBA)
Perfluorooctanoic acid (PFOA)
2,2,2-Trifluoroethyl Nonafluorobutanesulfonate (PFBS)
6:2 Fluorotelomer sulfonate (6:2 FTS)
8:2 Fluorotelomer Alcohol (8:2 FTOH)

In some embodiments, a filtration or separation medium comprising the covalently functionalized fluorographite as stationary phase can achieve greater than about 80 percent (%) of contaminant removal from a volume of liquid, greater than about 90% of contaminant removal from a volume of liquid, greater than about 95% of contaminant removal from a volume of liquid, or up to about 100% of contaminant removal from a volume of liquid.

The filtration medium can be regenerated by any process not inconsistent with the technical objectives described herein. In some embodiments, for example, the stationary phase can be regenerated via NaCl solution or brine solution. In some embodiments, the separation medium does not exhibit performance losses for contaminant removal after at least 20 or 30 regeneration cycles. In some embodiments, for example, the separation medium loses less than 1%, less than 0.5% or less than 0.1% contaminant adsorption performance over at least 40 cycles of regeneration.

In another aspect, methods of functionalizing fluorographite are provided. A method, in some embodiments, comprises providing a reaction mixture including fluorographite and an oligomeric species or polymeric species. Surfaces of the fluorographite are covalently functionalized with the oligomeric species or polymeric species via a radical reaction mechanism. The oligomeric species or polymeric species, for example, can comprise a radical end prior to covalently binding to sites of defluorination. Moreover, the reaction mixture can comprise an aqueous continuous phase.

### II. Covalently Modified Cellulose Nanofibers

In another aspect, nanocomposite materials based on cellulose nanofibers are described herein. In some embodiments, a nanocomposite material comprises oligomeric chains or polymeric chains covalently attached to surfaces of cellulose nanofibers. The oligomeric chains or polymeric chains, for example, can be in spacings or locations in between individual cellulose nanofibers. In some embodiments, the cellulose nanofibers have individual diameters of 5 nm to 50 nm.

As described above, the oligomeric chains or polymeric chains can comprise cationic, anionic, or fluorinated moieties. Chemical identity of the oligomeric chains or polymeric chains can be selected on the type of contaminant to be removed from a water source. The oligomeric chains and polymeric chains can have any identity and/or properties described in Section I above. Moreover, separation media comprising cellulose nanofibers covalently functionalized with oligomeric chains or polymeric chains can remove from a water source any of the contaminants described in Section I above, including NOM, heavy metals, and PFAS substances. The covalently functionalized cellulose nanofibers can also exhibit the removal efficiencies recited in Section I above.

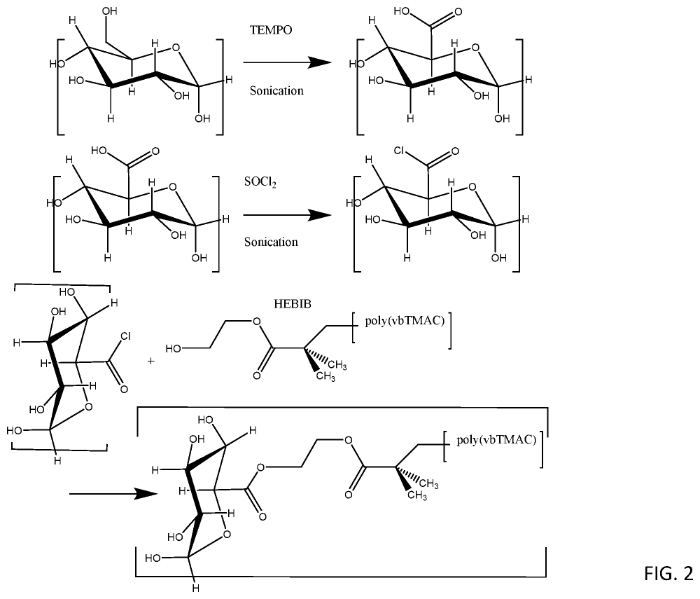

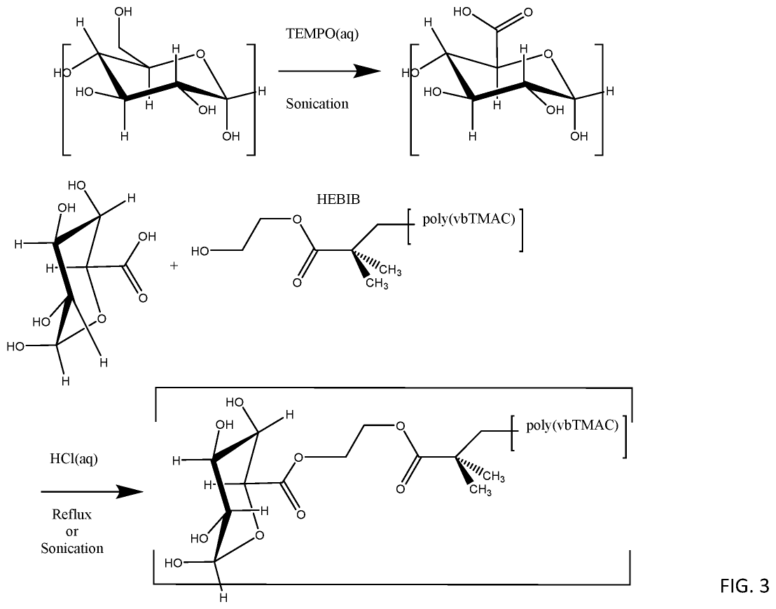

In another aspect, methods of making cellulose-based nanocomposite materials are described herein. A method, in some embodiments, comprises providing cellulose nanofibers, and oxidizing the nanofibers to provide reactive surface sites. Oligomeric species or polymeric species are provided and covalently attached to the reactive surfaces sites via esterification or a radical reaction mechanism. In some embodiments, the esterification or radical reaction mechanism occurs in an aqueous reaction medium. FIG. 2 and FIG. 3 illustrate methods of producing cellulose nanofibers covalently functionalized with oligomeric chains or polymeric chains via esterification, according to some embodiments. In some embodiments, a radical reaction mechanism for covalent functionalization of the cellulose nanoparticles comprises amidating reactive carboxylate surface sites with allyl amine or vinyl amine. The oligomeric chains or polymeric chains are subsequently attached to the allyl amine or vinyl amine via radical reaction, such as atom transfer radical methods.

These and other embodiments are further illustrated in the following non-limiting examples.

### Example 1—Covalently Functionalized Fluorographite and Associated Separation Media

In this example, the defluorination of fluorographite within an all-aqueous environment, utilizing the living radical chain end of short strands of polyelectrolyte at neutral pH is demonstrated. Defluorination below 10 at. % was achieved using this green and sustainable method. Radical centers on fluorographite can interact with electron-rich or electron-donating species. Delocalization of spin centers due to the presence of C—F σ* orbitals in the neighboring atoms can stabilize these Singly Occupied Molecular Orbitals (SOMO). These surface defects can act as the initiation sites of defluorination which eventually increase upon reaction with electron-rich species. Therefore, defluorinated carbons will develop a slight positive charge due to highly electronegative fluorine atoms bound to neighboring carbon atoms. These defluorinated sites are potential centers for polyelectrolyte radical attack and subsequent covalent attachment. The number of spin centers increases during the reaction, resulting in a well-functionalized polyelectrolyte functionalized fluorographite (PFG) resin material.

The PFG resins designed in this study were particularly well suited for removing environmental contaminants of emerging concern that have been highlighted by the United States Environmental Protection Agency (US EPA). Per- and polyfluoroalkyl substances (PFAS) have notoriously high thermal and chemical stability, making them resistant to environmental degradation. Therefore, these persistent and toxic impurities are pervasive throughout natural water systems. Recent studies have shown the presence of PFAS in human blood serum, breastmilk, human urine, human embryonic fetal organs, making PFAS emerging threats to the environment, and human health. The emerging threat suggests the need for increased regulation. The maximum contaminant level (MCL) of PFAS set by the US EPA is 70 ng L⁻¹. Diatrizoic acid (DTZA) is one of the iodinated X-ray contrast media (ICM) that are used for medical imaging. ICM are found in natural water resources in the range of 100 μg L⁻¹ and conventional wastewater treatment systems are not effective in removing them. Oxyanions of As, W, V, Cr, and other species are commonly found emerging metal contaminants in wastewater streams. Metalloid oxyanions are non-biodegradable, highly water-soluble, and are extremely mobile species that are also deleterious to human health and the environment. All these contaminants have been specified by the US EPA as contaminants of emerging concern.

PFG materials described in Section I above and further illustrated in this example have been designed with the motivation of removing these US EPA identified emerging contaminants. The PFG resins in this study have demonstrated efficient, high capacity removal of contaminants during short contact time and high water flux. The present PFG materials have demonstrated strong binding interactions with the compounds studied. Due to the higher accessibility of binding sites, high absorption capacity is demonstrated with rapid binding kinetics. The PFG resins have achieved ≥90% removal of all tested contaminants at environmentally relevant concentrations, and these PFG resins are easily regeneratable and readily reusable without any significant performance degradation. Hence, PFG resins provide an efficient platform for tailoring graphene derivatives with a high degree of functionalization toward sustainable industrial applications.

Synthesis, morphological and physicochemical characterization of poly(vbTMAC) functionalized fluorographite (PFG): Fluorographite is an insulator with highly stable C—F bonds with bond dissociation energies larger than 418 kJ mol⁻¹. In the present example, the surface modification and functionalization of fluorographite was investigated in the presence of grown (>95% conversion) polyelectrolyte brushes generated by activators regeneration by electron transfer assisting atom transfer radical polymerization (ARGET ATRP) in water under neutral pH conditions.

In Table 2, the defluorination results are summarized for some of the materials characterized in this study. Specific synthetic details are described below and in the supplementary information. As purchased pristine fluorographite contained 58 At % F and a small amount of N (samples were stored in air and not degassed before X-ray photoelectron spectroscopy (XPS)). The reaction of fluorographite with polyelectrolyte results in a decrease of F At %. Many control experiments were carried out to further specify the role of the living end on the polymer strand. When pristine fluorographite is refluxed in pure degassed water for 3 days, the material turns black but there is very little defluorination (F At %=56%). When pristine fluorographite is vigorously stirred in degassed water at room temperature for three days the material stays white and is not defluorinated, as expected.

TABLE 2
Elemental composition (in terms of At %) of
fluorographite, fluorographite under reflux
(control studies) PFG resins analyzed by XPS.

| | Element | | |
|---|---|---|---|
| Material | Carbon | Fluorine | Nitrogen |
| Pristine | 40.76 | 58.76 | 0.49 |
| Fluorographite | | | |
| Refluxed | 43.51 | 56.03 | 0.46 |
| Fluorographite | | | |
| (control) | | | |
| PFG-I | 61.96 | 36.14 | 1.90 |
| PFG-V | 82.69 | 14.50 | 2.81 |
| PFG-V+ | 87.78 | 9.55 | 2.66 |

Sonicating pristine fluorographite in cold degassed water the material stays white and remains stuck to the glass without dispersing in water. The addition of polyethylene glycol methyl ether (PEG), a hydrophilic polymer, at the same concentration and molecular mass of our polyelectrolyte increases dispersion of fluorographite as the physisorbed polymer acts as a surfactant around fluorographite particles but does not result in defluorination (even after 3 days at reflux in degassed water). Control experiments on pristine fluorographite in water with ARGET-ATRP reaction reagents (reducing agent, or catalyst) both at cold temperature and at reflux did not result in any significant reduction of the F At % as detected by XPS or EDX. It was concluded that low-temperature sonication alone, high-temperature reflux, the presence of ARGET ATRP reaction reagents, or the addition of soluble polymers without radical ends, do not initiate the defluorination process. Whereas the addition of our living end poly(vbTMAC) does result in exfoliation, stabilization, and defluorination of the fluorographite material.

PFG-I was the only material that included the radical initiator TEMPO during the polyelectrolyte functionalization. The material clearly defluorinates, as expected, to F At %=36. The synthesis of PFG-V does not contain any added TEMPO. PFG-V and PFG-V+ are green aqueous processes that include only fluorographite and poly(vbTMAC) in water. After 2.3 h of sonication and three days of reflux, PFG-V defluorinated to F At %=14.5%. PFG-V+ was sonicated under Ar(g) for 26 hours followed by reflux for three days. This resulted in a slightly lower F At %=9.6%. Future studies will measure the kinetics of defluorination and are not the subject of this study. XPS measurements on all of the PFG materials (PFG-I through PFG-V) show a small increase in the At % of N, consistent with the functionalization with poly(vinylbenzyl-trimethylammonium chloride).

Fluorographite does not form stable dispersions in water but after defluorination and functionalization in presence of polyelectrolyte, it forms a stable black aqueous dispersion. The incoming polyelectrolyte radicals attack the F atoms on fluorographite and generate spin centers. Spin centers are a good source of polyelectrolyte functionalization centers. Two spin centers adjacent to one another can result in the formation of C═C bonds. The generation of graphitic regions is detected in XPS analysis, where the deconvolution of the carbon peak of the PFG resin exhibited an intensified C═C bond peak (FIG. 1d). Additionally, functionalization with styrenic polymers will also add to the detection of C═C bonds. Hence, thermogravimetric analysis (TGA) was performed to measure the degree of polymer functionalization discussed below.

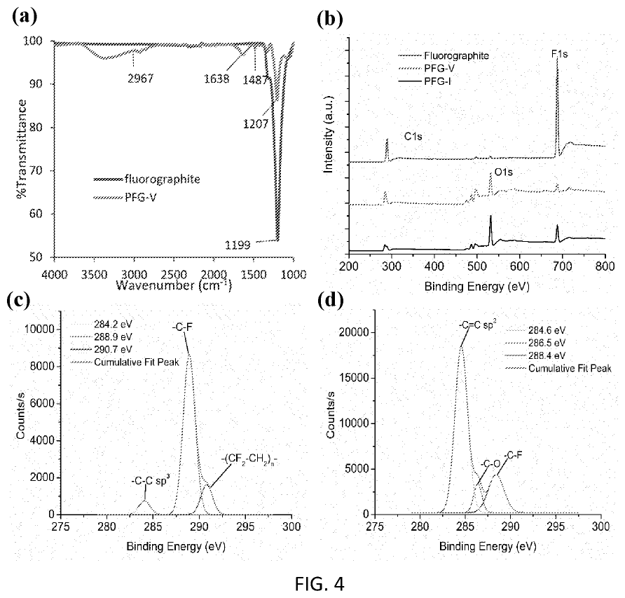

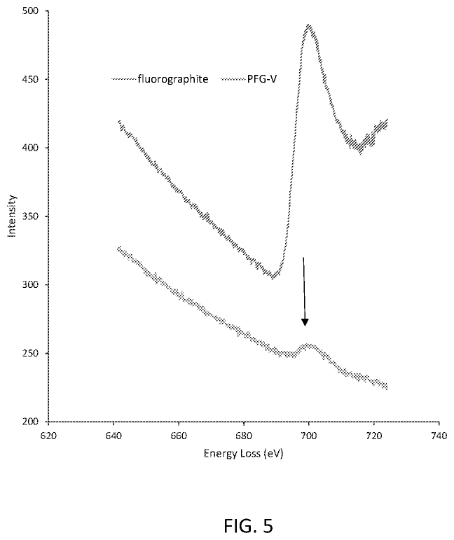

In the present example, defluorination and functionalization of fluorographite mediated by ARGET ATRP assisted polyelectrolyte in the presence of TEMPO (PFG-I) and the absence of TEMPO (PFG-V) were compared. Attenuated Total Reflectance Fourier Transform Infrared Spectroscopy (ATR FTIR) spectra of purified PFG-V resin confirmed the functionalization with polyelectrolyte strands of vbTMAC. IR bands at 1487 cm⁻¹ are consistent with the presence of C—N stretching vibrations from the quaternary ammonium group and the absorption at 1638 cm⁻¹ is consistent with the stretching mode of sp² hybridized carbon due to the increase in C═C of PFG-V, which is also confirmed by XPS analysis. Fluorographite exhibits a sharp C—F absorption stretch at 1199 cm⁻¹, while the C—F stretch of PFG-V was reduced and blue-shifted to 1207 cm⁻¹. The PFG-V C—H stretch at 2967 cm⁻¹ corresponds to the covalently attached poly(vbTMAC). Defluorination of PFG resins was confirmed with XPS analysis (FIG. 4B and Table 2). Comparison of the C 1s spectra of fluorographite and PFG-V show (FIGS. 4C and 4D) a decrease in intensity of the C—F bond peak at 288.4 eV for PFG-V. There is an appearance of a new peak at 284.6 eV due to the sp² hybridized carbon as a result of defluorination and the functionalization carbon in the styrene ring of vbTMAC. It was observed that PFG-V resulted in higher defluorination without the requirement of external radicals. Additionally, TEMPO is capable of capturing carbon radicals (i.e., polyelectrolyte radicals in our case) which may decrease the yield of active polymer radical chains in PFG-I synthesis. This analysis supports the case that we accomplished effective defluorination, a conclusion which is further supported by EELS analysis. By comparing the F—K edge of fluorographite with F—K edge of PFG-V at 700 eV (FIG. 5), a significant decrease in the intensity was observed, indicating defluorination.

TGA was used to investigate the thermal stability of purified PFG resins. Initially, all the PFG samples show 2-4% weight loss due to residual physisorbed water. Residual TEMPO was lost as the temperature rose to 125° C. and does not contribute to weight loss in the polymer or fluorographite in PFG-I samples. Between, 125° C. to 400° C., the initial mass loss is from the decomposition of poly(vbTMAC), due to styrene and quaternary ammonium decomposition, where the trimethylammonium chloride decomposes to trimethylamine and HCl gas. The second significant mass loss between 400-600° C. was due to loss of F from fluorographite/fluorographene in all PFG samples.

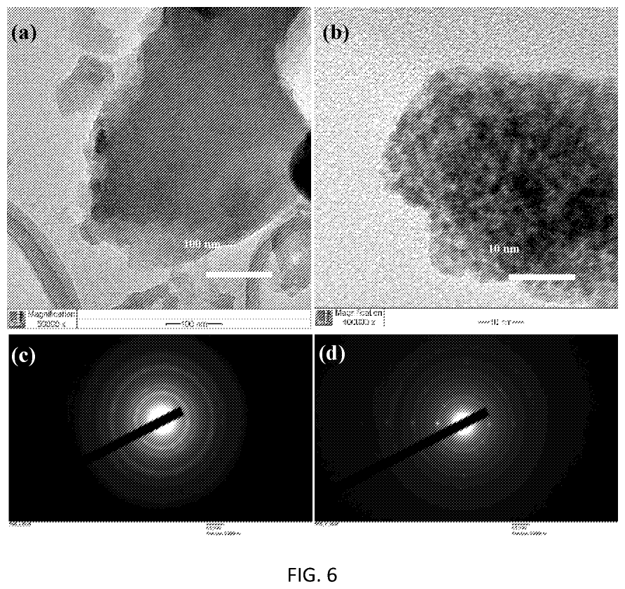

Transmission electron micrographs (TEM) of fluorographite and PFG-V were compared to evaluate differences in the morphology due to functionalization (FIGS. 6A-6D). Fluorographite shows the stacking of layers of large sheets (FIG. 6A), whereas PFG exhibits a different morphology. At 400,000× magnification, the small PFG crystallites exhibit dense functionalization of poly(vbTMAC) brushes of globular morphology (FIG. 6B). From previous work, the radius of gyration of 26-mer units of polyelectrolyte was measured to be 1.7 nm. The diameter of the globular structures at the surface of the fluorographene sheet is approximately 3 nm, suggesting that these are the polyelectrolyte brushes in highly functionalized domains. SEM images at 250,000×, also show globular/nodular polymer structures on the surface of the ellipsoidal nanoplatelet PFG crystallites.

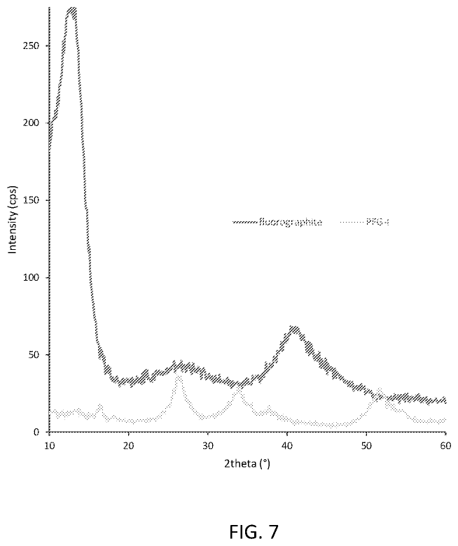

XRD analysis of PFG-I was compared with fluorographite. XRD of fluorographite (FIG. 7) shows a broadened (002) reflection peak at 2θ=26.8° compared to the more intense and sharp peak at 2θ=26.8° of the PFG-I sample. This is indicative of the resulting graphenic regions due to the defluorination of the fluorographite after polymer functionalization. This 20 value corresponds to d-spacing (or interlaminar spacing) of 0.33 nm (calculated from Bragg equation), which is consistent with the selected area electron diffraction (SAED) patterns of PFG-I and PFG-V obtained by TEM analysis. The (001) reflection peak at 2θ=12.9° corresponds to the hexagonal structure with high fluorine content in fluorographite. After polyelectrolyte functionalization (PFG-I), this 2θ=12.9° peak was significantly reduced, undetectable in XRD or SAED analysis. Another characteristic (004) reflection peak was prominent at 2θ=51.7° for PFG-I, which confirms the formation of the crystalline structure of graphite during defluorination. This peak corresponds to d-spacing of 0.18 nm which is supported by SAED patterns of PFG-I and PFG-V (FIGS. 6C and 6D). A fluorographite reflection peak at 2θ=41.0° was visible in the SAED pattern of PFG-V corresponding to a d-spacing of 0.22 nm. This (100) reflection peak corresponds to C—C in-plane length in the reticular system. From XRD analysis, there is a reflection peak at 2θ=33.9° for PFG-I, which corresponds to d-spacing of 0.26 nm. This measurement agrees well with SAED patterns of PFG-I and PFG-V (FIGS. 6C and 6D). Additionally, we observed that the diffusive rings of fluorographite are in the transition phase of conversion to the highly ordered crystalline phase in PFG resins, giving the impression of prominent rings in SAED analysis (FIGS. 6C and 6D).

The number of layers in graphitic materials can be calculated by combining Debye-Scherrer

$$( D = \frac{K λ}{βcosθ} )$$

and Bragg equation

$$( d = \frac{λ}{2 sin θ} )$$

resulting in

$$N = ( \frac{D}{d} + 1 )$$

where D is the average crystal height, d is the interplanar spacing, R is full-width half maxima and λ is the wavelength of the X-ray source. Considering the (002) reflection peak contributing to inter-layer spacing for PFG-I, K is a crystallite shape constant equal to 0.89 for spherical crystals with cubic unit cells,³⁹ β as 1.887°, λ as 0.154 nm, D and d was calculated to be 4.52 and 0.34 nm, respectively. This results in N being equal to 14 layers and when the number of layers is close to 10, they define graphitic properties. Hydrodynamic diameter and zeta potential of PFGs were measured using DLS Zetasizer. The measured hydrodynamic diameter of PFG-V was 76.4±2.4 nm. These particle sizes are consistent with size of graphene sheets from XRD, TEM, and SEM data. The measured positive zeta potential (+53.5±0.46 mV) confirms a conformal coating of polyelectrolyte onto the fluorographite surface.

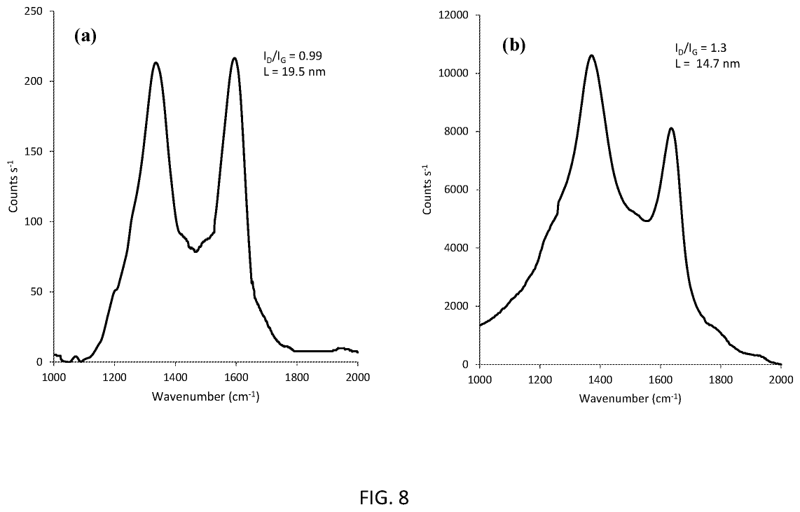

The functionalization density of SWCNTs has been shown to be proportional to the intensity ratio between D (I_{D}) and G band (I_{G}) (D:G ratio). However, since fluorographite is already covalently modified with F atoms attached to every C atom and we functionalize the defluorinated regions simultaneously with incoming polymer, the D:G ratio does not play an important role in determining the % functionalization in this case. Measured D:G ratios of fluorographite, PFG-I, and PFG-V were 0.99, 0.93, and 1.30, respectively. Therefore, Raman D:G ratio was not used here to quantify the functionalization density of polymer on the PFG nanostructure. Raman spectra of fluorographite and PFG-V are shown in FIG. 8. The 2D Raman peak is an important metric for understanding the exfoliation of graphite into graphene layers but due to the high functionalization of PFG-V, there is broadening of all of the Raman bands making it difficult to infer much information from the 2 D band (2717 cm⁻¹). Crystallite size (L) of graphitic materials can be calculated using the equation

$$L ( nm ) = ( 2.4 \times {10}^{- 10} ) λ {\frac{{I}_{D}}{{I}_{G}}}^{- 1}$$

where λ is the laser wavelength. The crystallite sizes are listed in Table 3.

TABLE 3
Crystallite sizes calculated from
Raman analysis using the formula
$L ( nm ) = ( 2.4 \times {10}^{- 10} ) λ \frac{{I}_{D}^{- 1}}{{I}_{G}}$

| Sample | I_{D} | I_{G} | L (nm) |
|---|---|---|---|
| Fluorographite | 213.4 | 216.6 | 19.5 |
| PFG-I | 5583.6 | 5967.9 | 20.3 |
| PFG-V | 10612.9 | 8113 | 14.7 |

Morphology of thin films, Adsorption Kinetics and Isotherm, Water Purification Testing:

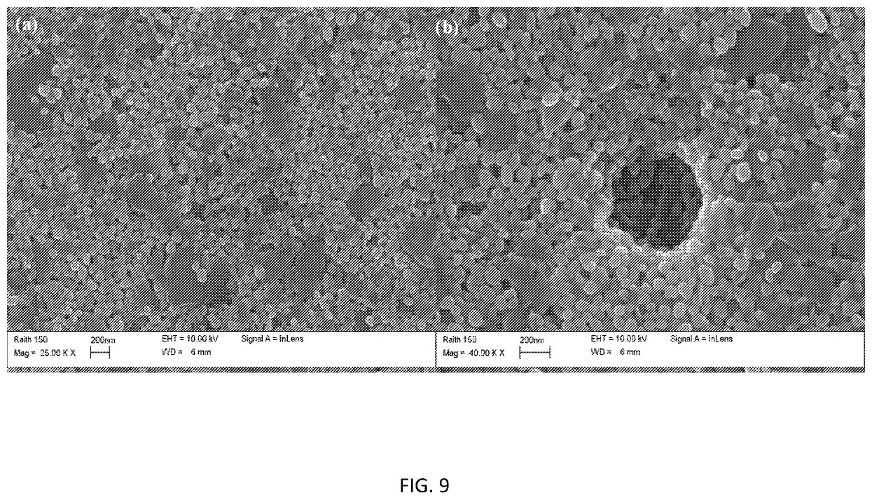

Morphology of PFG resin thin-film assembly is an important parameter of the structure-property-function relationship. We performed SEM analysis on thin films of PFG-V to understand how the assembly of graphitic layers contributes to effective target contaminant removal and high water flux (FIGS. 9A and 9B). It was observed that a 700 nm thick PFG film exhibited uniform horizontal stacking of nanoplatelets without pinholes or cracks. In FIG. 9B, the hollow artifact is due to a particle that was attached to the film during sample preparation which dislodged during processing. The artifact revealed a continuous stacking assembly of the crystallite layers with the bottom layer having a similar horizontal assembly as the top one.

Consistent with TEM analysis, the SEM image at 250,000× magnification also showed nanoplatelets conformally coated with polymer nodules. The measured diameter of these nanoplatelets (˜70 nm) agrees with the number distribution of the measured hydrodynamic diameter of these crystallites using DLS. Water flux values of PFG-V as shown in Table 4 are 10-50 times higher compared to the water flux of graphene oxide membranes of similar thickness at 1 atmospheric pressure. The assembly of nanoplates facilitates molecular transport through the tortuous path produced from stacking, resulting in high water flux. This tortuosity even allows molecules to be in proximity with the exposed quaternary ammonium ions during molecular transport which leads to fast “contact like” adsorption. A 3.3× decrease in water flux was observed when the areal mass density (mg cm⁻²) of the thin-film was increased by 5 times, consistent with a uniform thin-film without cracks or pinholes.

TABLE 4
Water flux measurements of PFG-V thin films deposited
on MCE determined at specific pressure. Multiple
runs were measured and averaged.

| Mass density | Water flux |
|---|---|
| (mg cm⁻²) | (L h⁻¹m⁻²bar⁻¹) |
| 0.094 | 3724 ± 519 |
| 0.469 | 1135 ± 9 |

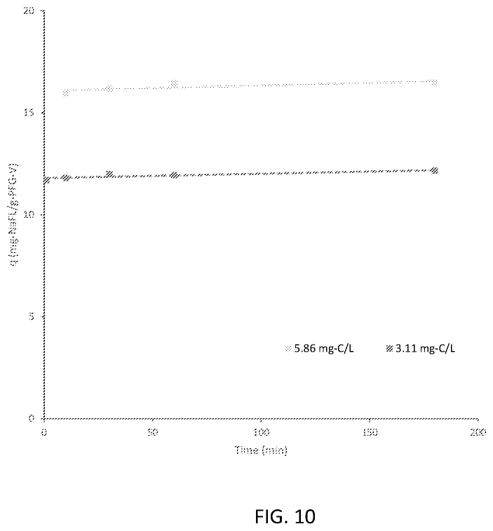

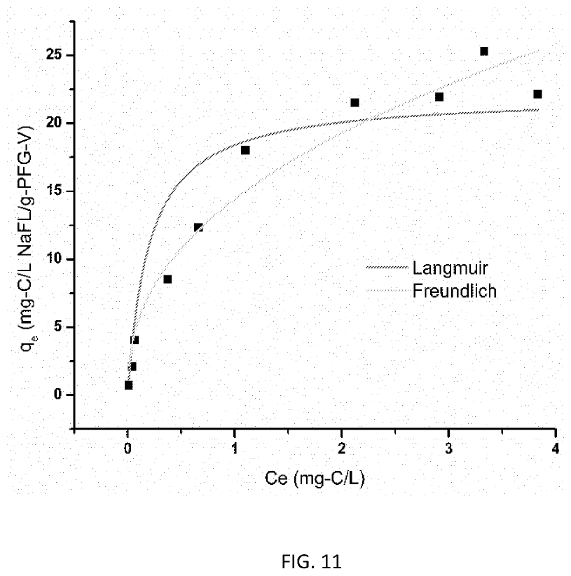

Adsorption kinetics of PFG-V was plotted as shown in FIG. 10. Extrapolating the trendline, the measured pseudoequilibrium “qₑ” is achieved within a few seconds. These PFGs are open resins, without polymer cross-linking, and with large specific surface area allowing for highly accessible binding sites. The adsorption isotherm behavior of PFG materials were measured and analyzed where we have plotted adsorption loading, q as a function of equilibrium concentration, Cₑ (as shown in FIG. 11). Adsorption isotherm curves of PFG resins were fit using Langmuir and Freundlich models. According to Akaike's Information Criterion test (AIC), the Freundlich model demonstrated better fitting with an AIC of 21.4 compared to Langmuir with AIC of 31.6 in PFG-V. Non-linear fitting of these data to the Freundlich is

$${q}_{e} = {K}_{f} {C}_{e}^{\frac{1}{{n}_{f}}}$$

that allows extraction of K_{f} and 1/n_{f} which represent adsorption capacity and adsorption binding strength, respectively. We measured and adsorption capacity of K_{f}=14.4±0.8 (mg/g)(L/mg) for the PFG materials which is significantly greater than that from the published graphene oxide materials (K_{f}=0.763−1.7 (mg/g)(L/mg)).⁴⁸ The binding strength of our PFG materials is also stronger with 1/n_{f}=0.4 as compared to the graphene oxide membrane materials (1/n_{f}=0.6),⁴⁸ where smaller 1/n_{f} is consistent with stronger binding at low contaminant concentration. Control studies were performed to analyze the sorption ability of fluorographite. 2 ml of 3.00 mg-C/L of NaFL was incubated overnight with 1.0 mg of fluorographite and the sample was filtered through 13 mm wide 0.2 μm pore size MCE membranes after 24 hours and analyzed using UV-vis-NIR Spectrophotometer. It resulted in negligible removal (0.60 mg-C/L).

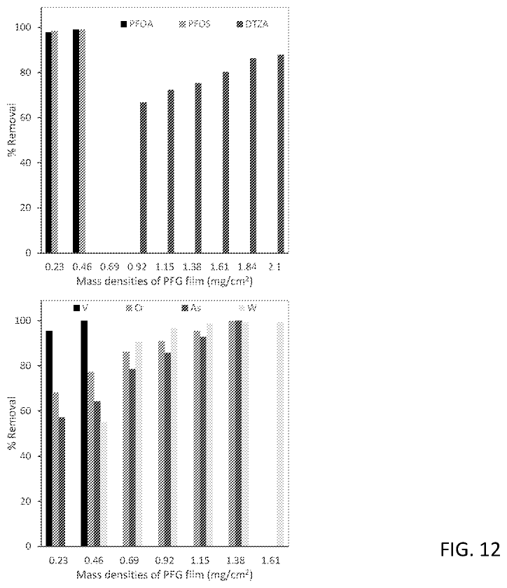

For adsorption capacity studies, thin films of the covalently functionalized fluorographite of the present example were prepared onto a mixed ester cellulose (MCE) support. and employed them for fast filtration removal of contaminants. as opposed to incubation studies that have been explored extensively by researchers to perform adsorption studies with porous materials that do not have high open resin surface area like our materials. PFOS and PFOA removal was tested with a stock concentration of 95.64 μg L⁻¹ and 95.02 μg L⁻¹, respectively and achieved >99% removal using a PFG-V thin film deposited (700 nm thick, 0.46 mg cm⁻¹) on MCE support as measured by EIS-MS (method described below). The fast-filtration method removed the contaminant is a few seconds of film exposure and has a water flux of 1135 L m⁻²h⁻¹bar⁻¹ at one atm pressure (FIG. 12 upper panel). Other materials discussed above do not need long incubation times for this level of removal. The open resin microstructure of the PFG materials enables the high binding site accessibility. PFG-V resins have partial fluorophilicity due to <15 At % F which supports faster and effective removal of PFAS. We were able to remove DTZA up to 88% using an analyte concentration of 117 μg L⁻¹ due to the π-π* interactions with PFG-V (FIG. 12 upper panel). The method detection limit (MDL) of all the contaminants screened using the MS method was below 1 μg L⁻¹ (ppb).

The removal of metal oxyanions CrO₄²⁻, VO₄³⁻, WO₄²⁻ and H₂AsO₄¹⁻/HAsO₄²⁻ using PFG-V resins was also measured. MDL values in accordance with ICP OES and MCL values of these metal ions are specified by US EPA. There is no MCL specified for W by US EPA but as a reference, we are considering the standard established by the Occupational Safety and Health Administration of 3 μg L⁻¹ as 15-minute short term exposure limit for airborne exposure to soluble tungsten. Stock concentrations of these oxyanions were prepared above the MCL limit but within the range of environmentally relevant concentrations. A facile >99% removal of all oxyanions was achieved with filtrate concentrations reaching well below MCL (FIG. 12 lower panel).

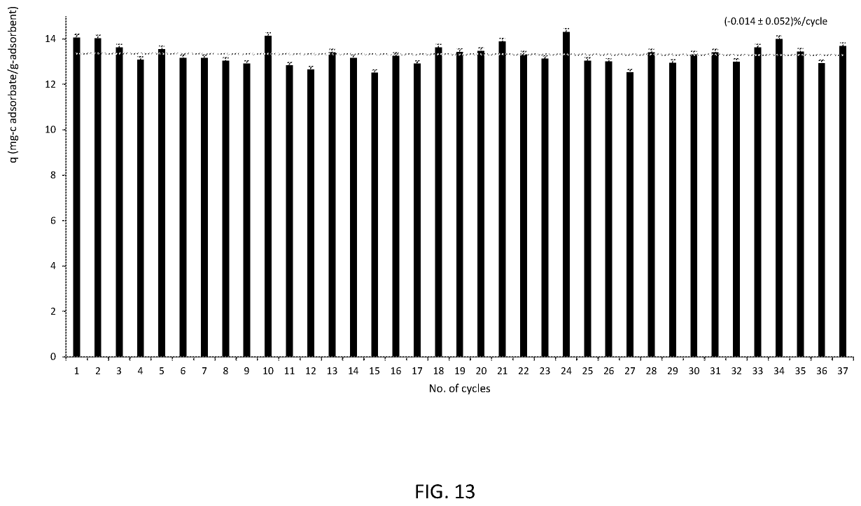

The regeneration behavior of PFG-V resins was measured by using a vial incubation protocol. Each cycle consists of incubating PFG-V films in surrogate contaminant (NaFL) until pseudoequilibration is reached so that the loading q, can be measured. The film was then rinsed with brine and then water to complete the cycle. Regeneration studies demonstrated a slope of −0.014±0.052% per cycle after 37 adsorption-desorption cycles, as illustrated in FIG. 13. Without measurable loss of functionality, it is projected that these materials can be regenerated and reused for over 1000 cycles leading to a sustainable water purification solution. PFG resins have a unique structure that supports faster kinetics, high percent removal, and high water flux. These materials have been fabricated keeping small systems of thin membranes in mind and extensive structural studies incorporating several cycles of breakthrough studies have not been studied.

In summary, aqueous synthesis of defluorination and subsequent functionalization of fluorographite was performed to obtain functional graphitic materials for water purification purposes. This 2D material demonstrated a balanced structure-property-function relationship with high percent removal (>90%), easily achieved at high membrane water flux (1135 L m⁻²h⁻¹bar⁻¹) and faster kinetics due to surface functionalization as compared to other porous materials. PFGs demonstrated higher performance than graphene oxide analogs and other materials synthesized using caustic/toxic conditions. Kinetics of analyte adsorption using PFG resins were rapid (≤one minute) and demonstrate high loading capacity with K_{f}=14.4±0.8 (mg/g)(L/mg) due to open resin structure. Transmission electron micrographs revealed crystalline nanoplates with poly(vbTMAC) functionalized domains. These resins were capable of high percent removal of emerging pervasive analytes under environmentally relevant concentrations well below their MCL. Therefore, highly functionalized PFG materials are compelling models for next-generation high capacity removal of contaminants of emerging concern and other sustainable technology applications.

### Chemicals and Materials:

All reagents were used as purchased without additional purification or modification. Graphite, fluorinated, polymer (Fluorographite, >61 wt % F) (Aldrich, Lot #MKCJ1629) was used. Polymer synthesis and functionalization chemicals: vinylbenzyl trimethylammonium chloride (vbTMAC) (Fisher, 97%; Lot #A0311318) monomer, copper(II) bromide (Acros, 99+%; Lot #A0344238), tris(2-pyridylmethyl)amine (TPMA) (TCI,>98.0%; Lot #Z8GMO-AD) for the catalyst, stannous octoate (SnOct) (Sigma-Aldrich, 92.5-100%, Lot #SLBP5072V) for the reducing agent, 2-hydroxyethyl 2-bromo-isobutyrate (HEBiB) (Sigma-Aldrich, 95%, Lot #MKBW2607) as the initiator. 2,2,6,6-tetramethylpiperidine-1-oxyl (TEMPO) (Sigma Aldrich, Lot #BCBZ3312) is the external radical. Other analytes include sodium chloride (Mallinckrodt Chemical, Lot #E42589), and disodium fluorescein (NaFL) (Sigma, Lot #BCBR1213V). Contaminants include perfluorooctanoic acid (PFOA) (Aldrich, Lot #MKCC6736), Potassium perfluorooctanesulfonate, 98% (PFOS) (Matrix Scientific, Lot #M22Q), Diatrizoic acid (DTZA) (Alfa Aesar, Lot #W24F030). Contaminants in the form of metalloids oxyanions of interest include Potassium Chromate (CrO₄²⁻) (Alfa Aesar, Lot #9186184), sodium orthovanadate (VO₄³⁻) (Alfa Aesar, Lot #Q21G501), Ammonium Tungstate (WO₄²⁻) (Alfa Aesar, Lot #227051), Arsenic (V) oxide hydrate (H₂AsO₄¹⁻/HAsO₄²⁻) (SPEX CertiPrep, Lot #25-72AS5M). Control studies included Poly(ethylene glycol) methyl ether (Sigma Aldrich, Lot #B2BR0088V).

### Workup and Purification

Workup and purification of the synthesis required: polypropylene membrane filters (0.45 m pore size, 47 mm wide, Lot #2075-5), nitrocellulose mixed ester (MCE) (Advantec, Lot. #70419200) with 0.2 m pore diameter and 13 mm width and (Advantec, Lot. #61202200) with 0.2 0.2 μm pore diameter and 25 mm width, 2 kDa MWCO dialysis membrane (Spectrum Labs, Lot. #3294218) 45 mm flat width and 29 mm diameter, 15 ml polypropylene centrifuge tubes (Corning®, Lot. #430052) and 50 kDa MWCO dialysis membrane (Spectrum Labs, Lot. #3292110) 34 mm flat width and 22 mm diameter. Membranes were activated by incubating for 10 min in Milli-Q water under constant stirring. Microcon® Centrifugal Filters (YM-100 lot #R1CN67003).

### Synthetic Methods.

### Aqueous Synthesis of Polyelectrolyte Functionalized Fluorographite in Presence of External Radical, Catalyst Complex, and Reducing Agent Using Sonochemistry and Reflux (PFG-I)

PFG-I was synthesized in a one-step process. Polyelectrolyte of vbTMAC grown using ARGET ATRP process as discussed in our previous publications to attain >95% conversion. In this case, molar ratio between vbTMAC (1.60, 7.57 mmol) and HEBiB (31 μL, 214 μmol) was 35. Copper(II) bromide and tris(2-pyridylmethyl)amine (TPMA) were used to form catalyst complex, and stannous octoate (SnOct) was used as reducing agent for ARGET ATRP mediated synthesis.^{32, 42} 9.0 mL aliquot of polymer reaction mixture (16.1 mL) was extracted and injected into a scintillation vial containing fluorographite (15.1 mg). Because fluorographite is superhydrophobic, its dispersion in aqueous media challenging. This mixture was sonicated using a sonication probe in a 10° C. bath (RTE-9 Endocal refrigerated circulating bath with coolant) at 72 W cm⁻² for one hour. During this period, the mixture was removed from the setup and manually shaken every 15 minutes at most to get the flakes of fluorographite into the aqueous phase. These steps aided in intercalation and physical wrapping of polyelectrolyte in between the fluorographite sheets, resulting in better dispersion. After one hour, the mixture was added back to the Schlenk flask (SF) and the vial was rinsed with MilliQ-water to obtain most of the fluorographite particles stuck to the glass surface and added to the reaction mixture making a final volume of 22 mL. The mixture was sparged for 10 min, sealed, and sonicated at 100 W cm⁻² for another 20 min. After 20 min, ˜75 mg of external radical TEMPO were directly added into the reaction mixture and sonicated for one more hour at 100 W cm⁻². The sonication probe was removed, the reaction mixture was sparged for 20 minutes, sealed, and then heated to reflux in a 110° C. oil bath for different time durations (one or three days) under Ar(g) positive pressure. As the reaction proceeds, the color of the reaction mixture changes to darkish grey. After the required duration, functionalization is stopped by removing the heat and exposing it to air. The reaction mixture was purified using the same steps as demonstrated in our previous publications.^{32, 42} The dispersion was centrifuged (200,000 g, one hour, 20° C.) in high ionic strength (4 M NaCl(aq)) to mechanically disrupt physisorbed polymer, and the sediments were collected, and this process was repeated until the concentration of unbound polymer in the supernatant goes below the detection limit.

PFG-V was synthesized using the purified polyelectrolyte described above. The polymer (16.6 mL) was sparged with Ar(g) for 15 min, and 9.0 mL was injected into a scintillation vial containing 15 mg of fluorographite. This mixture was ultrasonicated using a probe sonicator with the vial immersed in an ice water bath at 72 W cm⁻² for one hour and manually shaken once every 15 min. The hydrophobic fluorographite climbs up the walls of the vial until it becomes sufficiently coated with polymer. After one hour, the mixture was added back into the Schlenk flask, and the vial was rinsed with MilliQ-water to transfer most of the fluorographite particles into the reaction mixture making a final volume of 22 mL. This dispersion was sparged for 10 min, sealed, and sonicated at 100 W cm⁻² for 1 h 20 min at 100 W cm⁻². The sonication probe is removed, the reaction mixture is sparged for 20 more minutes, sealed, and then heated to reflux in a 110° C. oil bath for 3 days under Ar(g) positive pressure. As the reaction proceeds, the color of the reaction mixture changes to dark grey, which is a visual indicator of the defluorination process. After the required duration, functionalization is stopped by removing the heat. The reaction mixture is purified using the same steps demonstrated in our previous publications.^{32, 42}

PFG-V+ was synthesized and purified under the same conditions as PFG-V except that the reaction was scaled up using 4.35 mL of polymer (0.500 g), and 157 mg of fluorographite

TABLE 5
Overview of reagents/processes for various PFG syntheses

| Reagents | PFG-I | PFG-II | PFG-III | PFG-IV | PFG-V | PFG-V+ |
|---|---|---|---|---|---|---|
| TEMPO | + | − | − | − | − | − |
| Catalyst/ligand | + | + | + | − | − | − |
| complex | | | | (<5 ppm) | (<5 ppm) | (<0.1 ppm) |
| Reducing agent | + | + | − | − | − | − |
| Ultrasonication | 2.3 | 2.3 | 2.3 | 0 | 2.3 | 26 |
| time(h) | | | | | | |
| Reflux time (day) | 3 | 3 | 3 | 3 | 3 | 3 |

into 200 mL of water into a Schlenk flask without temperature control. The solution was sparged with Ar(g) for 30 min and ultrasonicated under an Ar(g) blanket at 100 W cm⁻² (500 W cm⁻² at ⅕ duty cycle) for 26 h. This was followed by the standard 3 day reflux. An overview of the rest of the processes are listed in Table 5.

### Characterization Methods.

Dynamic Light Scattering: Hydrodynamic diameter and zeta potential of various functionalized PFG batches were measured using Malvern DLS Zetasizer instrument. 10 ng mL⁻¹ of PFG sample solutions were used for DLS measurements. For Zeta measurements, 2 mg L⁻¹ of samples were used. An equilibrium time of 15 minutes was allowed in a normal room temperature sample cell. All results were reported as value±standard error, 95%, and outliers were removed.
Attenuated Total Reflectance Fourier Transform Infrared Spectroscopy: ATR FTIR spectrometer (PerkinElmer, Spectrum 100) was used to evaluate the presence of constituents of the PFG and fluorographite materials.
Raman Spectroscopy: Samples were prepared by drop-casting onto silicon wafers as a thin film for all surface enhanced Raman scattering measurements (XploRA Raman confocal microscope system, J Y Horiba, Edison, N.J.). Raman spectrometer with an excitation wavelength of 532 nm was used to analyze the relative integrated area under D band (sp³ hybridized carbon), G band (sp² hybridized carbon), and the 2D band of different samples between 600-3000 cm⁻¹. Peak frequencies were calibrated with silicon at 520 cm⁻¹ before each use. Data were analyzed and optimizations, like baseline and noise corrections, were performed using LabSpec 6 software. We have performed control studies in previous work, that provides evidence that ultrasonication has very little effect on D:G ratio under similar sonication duration.⁴²
X-Ray Photoelectron Spectroscopy: Samples were prepared by drop casting PFG sample solutions on Si chips as a thin film. For X-Ray Photoelectron spectroscopy (XPS, EscaLab 250Xi) analysis, monochromatic (1487.6 eV) Al Kα-ray source was used to probe the surface, with a spot size of 250 μm, and a flood gun was used to limit charging at the surface of the materials. Advantage Processing software package was used to analyze all XPS spectra.
X-Ray Powder Diffraction: Crystal phase of PFGs were analyzed using X-Ray Powder Diffraction (XRD, PANalytical (Netherlands) X′Pert PRO). XRD patterns were measured over a 20 range of 5-60 having a step size of 0.1000 with 10.0 s per step and scan rate of 0.010° per step.
Transmission Electron Microscopy: The morphology of the samples was analyzed by transmission electron microscope (TEM, JEOL JEM-2100Plus, acceleration voltage: 120 kV). For sample preparations, known concentrations of required dispersions were bath sonicated for five minutes at 5° C. with occasional manual shaking. Prepared carbon mesh TEM grids were dipped into the dispersion and dried overnight. For Electron Energy Loss Spectroscopy (EELS) analysis, emission current was set to 12 ρA, acquisition time was one second, energy filter aperture was 30 μm and illumination angle was set to 1.250 mrad. Selected area diffraction (SAED) were measured of all the samples deposited in TEM grids.
Thermogravimetric Analysis: Thermogravimetric analysis (TGA/SDTA851) of PFG, fluorographite, and TEMPO samples were done in Air mode. Mettler software was used to analyze all the measurements.

### Purification Testing Methods.

Waterflux studies: For water flux measurements, thin films of PFG-V of different mass densities (0.09 mg cm⁻², 0.46 mg cm⁻²) were used to evaluate the water flux using Milli-Q as permeate. A known mass of PFG was deposited on 25 mm wide 0.2 μm pore diameter MCE membranes as thin film and a known volume of Milli-Q was pushed through under vacuum with a known pressure. The duration for a known volume of Milli-Q to flow through was recorded, exact pressure was recorded and this process was repeated for several (>3) iterations. These thin films were dried for 30 minutes under vacuum and permeate flux was recorded again for several measurements. Water flux value was estimated from the following equation:

$${J}_{w} = \frac{Q}{AP △ t}$$

where J_{w} is water flux (L m⁻²h⁻¹bar⁻¹), Q is the volume of water (L), A is the surface area of the layers or film (m²), P is the pressure (bar) and Δt is the time (h) required for volume L of Milli-Q to flow through.
Kinetic studies: A known amount of PFG-V was added to microcentrifuge tubes holding a known concentration of an analyte, NaFL, and was vortexed for different time durations at 500 rpm at room temperature and neutral pH. At known time intervals, NaFL and PFG-V dispersions were pushed through 13 mm wide 0.2 μm pre diameter MCE membranes, and concentrations of filtrates were measured using UV-vis-NIR Spectrophotometer (Cary 5000, Agilent Technologies). The NaFL stock concentration was measured through control studies where the same amount of NaFL was pushed through a 13 mm wide MCE membrane without adsorbent. Control experiments were performed in the same conditions without the addition of adsorbents.
Adsorption Isotherm: A known amount of PFG-V material was added to the vial holding different known concentrations of an analyte, NaFL, which was vortexed for 4 h at 500 rpm at room temperature and neutral pH. Samples were filtered through 13 mm wide 0.2 μm pore size MCE membranes before analysis under UV-vis-NIR Spectrophotometer for adsorbent removal. Control studies were performed using fluorographite where a known concentration of NaFL was incubated for 24 h using a known mass of fluorographite powder and mixture was filtered and analyzed under UV-vis-NIR Spectrophotometer.
Analyte adsorption studies: We tested the removal of compounds under the US EPA emerging contaminants category, to evaluate the potential of our materials as smart point-of-use water purification systems that can remove contaminants of immediate concern. We demonstrated removal of perfluoroalkylated substances (PFAS) (viz., perfluorooctanoic acid (PFOA) and perfluorooctanesulfonate (PFOS)), iodinated X-ray contrast media (diatrizoic acid, DTZA)²¹ and oxyanions of metalloids specifically CrO₄²⁻, VO₄³⁻, WO₄²⁻ and arsenic (V) oxide hydrate (H₂AsO₄¹⁻/HAsO₄²⁻). Quantitative analysis of all oxyanions was performed using inductively coupled plasma atomic emission spectroscopy (ICP OES, 5100 Agilent California) and the remaining analytes were measured using liquid chromatography (LC) equipped with electrospray mass spectrometry (MS, Thermo Scientific, LTQ Velos Pro) using only the MS mode. The detections were operated with electrospray ionization in negative ion mode for PFOA and PFOS while in positive ion mode for DTZA with mobile phase A comprising of 0.5% formic acid in 100% LC-MS grade water and mobile phase B of 100% LC-MS grade acetonitrile. Calibration standards were prepared by serial dilutions with concentrations ranging from 1 μg L⁻¹ to 100 μg L⁻¹ for MS and 1 μg L⁻¹ to 1 mg L⁻¹ for ICP OES standards to formulate calibration curve. Calibration standards of all oxyanions were made using 2% (v/v) nitric acid from purchased stock standards with concentrations of 1000 mg L⁻¹ in 2% (v/v) nitric acid. Influent concentrations for each oxyanion solution for water purification testing was prepared by serial dilution method in Milli-Q from purchased stock standard with concentration of 1000 mg L⁻¹ in water. Calibration standard and influent solutions for PFAS and DTZA were prepared by serially diluting stock concentrations in Milli-Q. These stock concentrations (1-4 mg L⁻¹) were prepared by spiking Milli-Q with solute and vortexing the mixture at 500 rpm for one hour. Concentrations of filtrate for ICP OES and MS were calculated from standard concentration curves and limits of quantification of each compound were analyzed. Concentrations of all compounds were determined by linear least square regression model of standard concentrations. For MS and ICP OES, every sample was run in duplicates or triplicates. Blanks were run before and after sample runs in both the instrument measurements.
Regeneration Studies: A known mass of PFG-V was deposited onto 25 mm wide 0.2 μm MCE membrane and that membrane was placed in a glass vial. The film was allowed to incubate in a brine solution (1.5 mL, 2.0 M NaCl(aq)) for 5 min. Brine was disposed of and MilliQ was used to rinse the remaining brine (3 replicates, 1.5 ml). A known volume and concentration of NaFL(aq) was added and allowed to incubate for 5 min and the concentration of NaFL(aq) solution after incubation was measured using UV-vis spectroscopy. In the subsequent brine rinses, the NaFL saturated films were regenerated by incubating in successive brine solutions (1.5 mL, 2.0 M NaCl(aq)) until the concentration of desorbed NaFL solution dropped down to 0.02 mg-C L⁻¹.

### Example 2—Covalently Functionalized Cellulose Nanofibers and Associated Separation Media

### Step 1: oxidation of cellulose fibers to cellouronic acid using (2,2,6,6-Tetramethylpiperidin-1-yl)oxyl (TEMPO)

In a 50 ml Schlenk flask, NaBr (0.258 g, 2.50 mmol) and TEMPO (11.64 mg, 74.3 μmol) were added in 25 ml Milli-Q. Following this, cellulose nanofibers (0.5118 g, 3.16 mmol) were suspended in the mixture and pH was adjusted to 10-11 using 0.199 M NaOH. The mixture was cooled using RTE-9 Endocal refrigerated circulating bath with coolant. Oxidation was initiated when 8.5 ml NaOCl (8.5 ml, 13.9 mmol) was added maintaining the pH. Sonicating probe and thermocouple were immersed, and neck of round bottomed flask was sealed with parafilm. Sonication was performed at 10° C. at 63 72 W cm⁻² for two hours to exfoliate and debundle the oxidized fibers. Transparent clear was observed as the supernatant floating on top of water precipitate. The oxidation was quenched by adding ethanol.

The cellulose products were twice washed thoroughly with ethanol at 200,000 g at 4° C. at one hour for each iteration. Ethanol rinsing was followed by one acetone rinse under the same conditions. Supernatant from each centrifugation was discarded and the pellet was scraped out. The pellet was dried overnight at 50° C. and dried pellet was dispersed in 100 ml Milli-Q and sonicated at 45 W cm⁻² for 20 minutes. The dispersion was centrifuged at 200,000 g at 20° C. for 75 minutes and supernatant was collected.

### Step 2: Formation of Amide Linkage

Oxidized cellulose (25 mg, estimated to be around 13.8 μmol) was added to 2 ml Milli-Q in a scintillation vial and bath sonicated for 20 minutes at neutral pH. (3-dimethylaminoproyl)-N′-ethylcarbodimmide hydrochloride (EDC) (30.13 mg, 157 μmol) was added to the suspension and was stirred and bath sonicated at neutral pH for additional 15 minutes. pH was adjusted to 8 using 0.199 M NaOH and allyl amine hydrochloride (15 mg, 160 μmol) was added, and stirred and bath sonicated overnight for 24 h. The temperature of the bath for the entire reaction was maintained at 25° C. At the completion of the reaction, the resulting reaction mixture was dialyzed overnight against Milli-Q through a 2 kDa MWCO cellulose dialysis membrane in a Soxhlet extraction at reflux to remove any unreacted monomer and other reagents from the desired amide modified cellulosic nanofibers for 24 h.

### Step 3: Poly(Vinylbenzyl Trimethylammonium Chloride) Poly(vbTMAC) Functionalized Cellulose Nanofibers

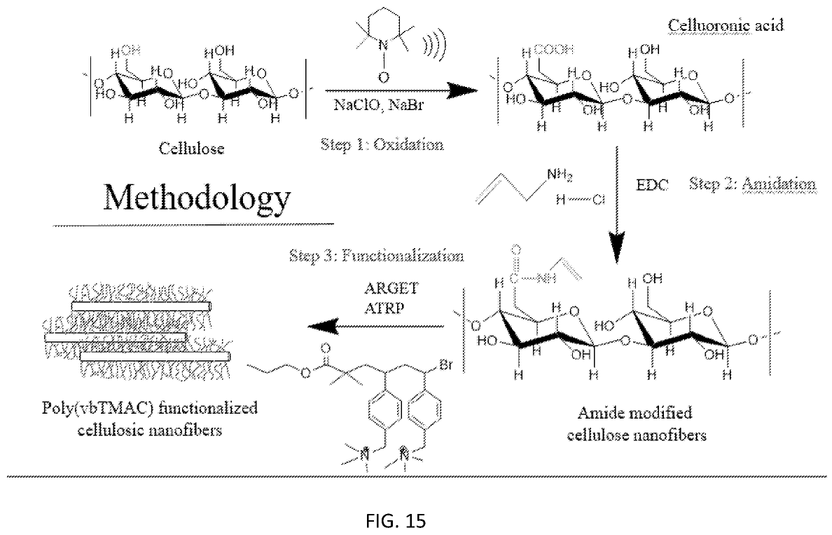

Cellulose nanofibers were covalently functionalized according to the scheme of FIG. 15. Polyelectrolyte of vbTMAC (Fisher, 97%; Lot #A0311318) was grown using ARGET ATRP process as discussed above to attain >95% conversion. Polymerization was performed in a Schlenk flask (SF). In this case, molar ratio between vbTMAC (1.60, 7.57 mmol) and HEBiB (31 μL, 214 μmol) was 35. Copper(II) bromide and tris(2-pyridylmethyl)amine (TPMA) were used to form catalyst complex, and stannous octoate was used as reducing agent for ARGET ATRP mediated synthesis. 9.0 mL aliquot of polymer reaction mixture (16.1 mL) was extracted and injected into a scintillation vial containing amide surface modified cellulose nanofibers solution which was already sparged for 10 minutes. The mixture was sonicated at 63 W cm⁻² for 15 minutes. The mixture was sparged for 10 minutes and added back into SF and the reaction was set to reflux for 24 hours.

After the required duration, functionalization is stopped by removing the heat and exposing to air. Reaction mixture was purified using the same steps as done in our previous publications. Dispersion was centrifuged (200,000 g, one hour, 20° C.) at high ionic strength (4 M NaCl(aq)) to mechanically disrupt physisorbed polymer, and sediments were collected and this process was repeated till the concentration of unbound polymer in the supernatant goes below the detection limit using UV-vis-NIR spectroscopy (ε254=0.0102±0.0007 (mg/L)⁻¹ cm⁻¹).

Purification testing: For adsorption testing, sodium fluorescein (NaFL) was used which is a surrogate to low molecular weight fulvic acids and exhibits strong absorbance at 490 nm (ε490=0.3576±0.0106 (mg-C/L)⁻¹ cm⁻¹. In a 8 ml vial, 2 ml of known concentration of NaFL was vortexed overnight with 0.2 mg of poly(vbTMAC) functionalized cellulose nanofibers and sample was filtered through 0.2 μm pore size 13 mm wide mixed ester cellulose (MCE) membrane before analysis under UV-vis-NIR Spectrophotometer for adsorbent removal and equilibrium loading (qₑ) was measured.

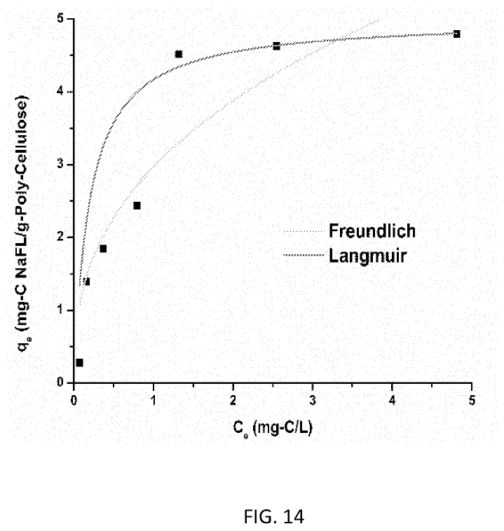

The hydrodynamic diameter of pristine cellulose nanofibers measured using dynamic light scattering (DLS) zetasizer was in the range of ˜250 nm. After purification, the successful debundling of —COO⁻Na⁺ modified cellulose nanofibers were confirmed using DLS with a hydrodynamic diameter of 24 nm. The oxidation of cellulose was confirmed using Fourier transform Infrared Spectroscopy with a strong peak around 1575.85 cm⁻¹ due to presence of —COO⁻Na⁺ group. Zeta potential of cellouronic acid solution was measured to be −28 mV. The amide reaction and functionalization of allyl amine hydrochloride was confirmed using Fourier Transform Infrared Spectroscopy (FTIR) where we observed shifting of C═O stretch at 1640.01 cm⁻¹, appearance of N—H in plane vibrations at 1534.6 cm⁻¹ and C—N stretch at 1429.6 cm⁻¹. Zeta potential of amide modified cellulose nanofibers were measured to be −19 mV. To confirm allyl amine hydrochloride is covalent attached to cellulosic scaffold, the reaction mixture was filtered using (μm) Anatop filter and found no peaks in the filtrate were detected which confirms there is no presence of any residual unreacted components. Zeta potential of poly(vbTMAC) functionalized cellulose nanofibers was measured to be +17.2 mV which confirmed the covalent attachment of polyelectrolyte to cellulose nanofibers. qe measured from incubation experiment was measured to be 12.6 mg-C NaFL/G polymer functionalized cellulose. Preliminary analysis of adsorption isotherm was performed to understand the adsorption behavior (FIG. 14).

Various embodiments of the invention have been described in fulfillment of the various objects of the invention. It should be recognized that these embodiments are merely illustrative of the principles of the present invention. Numerous modifications and adaptations thereof will be readily apparent to those skilled in the art without departing from the spirit and scope of the invention.

1. A nanocomposite material comprising:
oligomeric chains or polymeric chains covalently attached to surfaces of fluorographite at sites of defluorination.

2. The nanocomposite material of claim 1, wherein oligomeric chains or polymeric chains conformally coat individual platelets of the fluorographite.

3. The nanocomposite material of claim 1, wherein the oligomeric or polymeric chains are present in spacings between individual platelets of the fluorographite.

4. The nanocomposite material of claim 2, wherein the individual platelets of fluorographite each have thickness of 50 nm to 100 nm.

5. The nanocomposite material of claim 1, wherein the fluorographite comprises 5-20 individual platelets.

6. The nanocomposite material of claim 1, wherein the oligomeric chains or polymeric chains are not crosslinked.

7. The nanocomposite material of claim 1, wherein the oligomeric chains comprise 3-100 monomer units.

8. The nanocomposite material of claim 1, wherein the oligomeric chains or polymeric chains include one or more cationic moieties for anion exchange.

9. The nanocomposite material of claim 8, wherein the cationic moieties include quaternary ammonium groups or imadazolium groups.

10. The nanocomposite material of claim 1, wherein the oligomeric chains or polymeric chains include one or more anionic moieties for cation exchange.

11. The nanocomposite material of claim 1, wherein the oligomeric chains or polymeric chains comprise fluorinated moieties.

12. The nanocomposite material of claim 11, wherein the fluorinated moieties comprise fluorinated alkyl or fluorinated aryl moieties.

13. The nanocomposite material of claim 1, wherein the fluorographite comprises less than 40 at. % fluorine.

14. The nanocomposite material of claim 1, wherein the fluorographite comprises 5-20 at % fluorine.

15. A method of forming a nanocomposite material comprising:
providing a reaction mixture including fluorographite and an oligomeric species or polymeric species; and
covalently functionalizing surfaces of the fluorographite at sites of defluorination with the oligomeric species or polymeric species via a radical reaction mechanism.

16. The method of claim 15, wherein the reaction mixture comprises a continuous aqueous phase.

17. The method of claim 15, wherein the oligomeric species or polymeric species comprises a radical end prior to covalent functionalizing the sites of defluorination.

18. The method of claim 15 further comprising exfoliating the fluorographite prior to covalent functionalization with the oligomeric species or polymeric species.

19-50. (canceled)

51. A nanocomposite material comprising:
oligomeric chains or polymeric chains covalently attached to surfaces of cellulose nanofibers.

52. The nanocomposite material of claim 51, wherein the oligomeric or polymeric chains are present in spacings between individual cellulose nanofibers.

53. The nanocomposite material of claim 51, wherein the cellulose nanofibers have individual diameters in the range of 5 nm to 50 nm.

54. The nanocomposite material of claim 51, wherein the oligomeric chains or polymeric chains include one or more cationic moieties for anion exchange.

55. The nanocomposite material of claim 54, wherein the cationic moieties include quaternary ammonium groups or imidazolium groups.

56. The nanocomposite material of claim 51, wherein the oligomeric chains or polymeric chains include one or more anionic moieties for cation exchange.

57. The nanocomposite material of claim 51, wherein the oligomeric chains or polymeric chains comprise fluorinated moieties.

58. The nanocomposite material of claim 57, wherein the fluorinated moieties comprise fluorinated alkyl or fluorinated aryl moieties.

59.-72. (canceled)
